(19) United States
(12) Patent Application Publication
(10) Pub. No.: US 2008/0178899 A1
(43) Pub. Date: Jul. 31, 2008

(54) Foamable or sprayable hair styling product with polyalkoxylated silicone esters

(75) Inventors: Monika Moenks, Brunnenthal (CH); Jan Baumeister, Farvagny-le-Grand (CH); Sarah Maillefer, Middes (CH)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(73) Assignee: The Procter & Gamble Company, Cincinnati, OH (US)

(21) Appl. No.: 11/731,990

(22) Filed: Apr. 2, 2007

Related U.S. Application Data
(63) Continuation of application No. PCT/EP2005/009639, filed on Sep. 8, 2005.

(30) Foreign Application Priority Data
Oct. 15, 2004 (EP) 102004050211.0

(51) Int. Cl. A61K 8/89; A61K 8/04; A45D 7/04; A61Q 5/12

(52) U.S. Cl. 132/203; 424/47; 424/70.12

(57) ABSTRACT

Hair styling products are described that comprise a foamable or sprayable, gel-type emulsion, along with a device for spraying or foaming the emulsion. The emulsion contains esters of polyalkoxylated polydimethylsiloxanes, in particular esters of polyethoxylated polydimethylsiloxanes and fatty acids that have at least 8 C-atoms; a gelling agent or thickener, preferably carrageenan; emulsifiers, preferably alkoxylated silicone surfactants and water. Preferred embodiments are aerosol foams and pump sprays. The products can be used for conditioning and for styling of human hair.

### FIELD OF INVENTION

The object of the invention is a hair styling product that comprises a foamable or sprayable, gel-type emulsion and a device for spraying or foaming the emulsion, wherein the emulsion contains esters of polyalkoxylated polydimethylsiloxanes, gelling agents or thickeners, emulsifiers, and water.

### BACKGROUND OF THE INVENTION

Hair waxes are known hair treatment products. In particular, they can be used in order to provide a trendy shape to hair that is from short to middle length, and to lend hold, body, cohesiveness and shine to hair styles. Hair waxes also allow contours and textures to be produced in the hair style. The hair has a characteristic sensation by touch, a typical “waxy feel,” and since it possesses a persistent residual tackiness, the resultant restyling potential is a typical property of hair wax. Conventional hair waxes have a wax-like solid consistency and are usually offered in tubs. The following procedures are employed for their use: A quantity of the product is taken up with the fingers. A wax that is a solid at room temperature is spread over the palms of the hand, and is melted or at least significantly softened by the heat of the hands and by friction. This softening or melting enables the otherwise solid wax to be worked into the hair. In this softened or more or less liquid state, the wax is worked into the hair. Once in the hair, it cools and resumes its original solid consistency. In this way, the wax solidifies and the hair style that has been prepared will possess stability, hold, a typical waxy feel as well as an often slightly wet appearance. These procedures set narrow limits on the product performance for conventional styling wax products. In order to permit the wax to be worked into the hair readily, it should not be too hard when taken up into the hand, and the melting or softening point must be in the vicinity of body temperature. Otherwise, soft waxes of this type will only be able to achieve a moderate level of product performance with respect to the condition of the hair, and the hold and volume of the hair style. Nevertheless, the stress on the hair is comparatively high. A harder wax composition makes it possible to obtain much better cohesiveness and better hold, but the harder the wax, the harder will be the portion of the product that is used, and consequently the more difficult it will be to work with and to work into the hair. With a wax product in a tub, there are generally problems with poor dosability, conflicting requirements concerning the hardness of the wax product and the experience of the wax product having a disagreeable feel while it is being worked into the hair. Moreover, conventional products such as hair wax, wax foams, spray waxes, or styling foams are not yet completely satisfactory in their current form, particularly with regard to their application, and the strength and duration of their tackiness phase while still providing optimal styling potential.

The goal is to develop a product that exhibits the typical product performance of a solid hair wax, e.g., that provides the hair style with improved hold, body, and a typical waxy feel, and enables the restyling potential that is characteristic of hair wax and imparts to the hair a natural, semi-matte shine, while at the same time exhibiting a satisfactory tackiness phase, is readily spread and worked into the hair, can guarantee simple and clean handling, elicits innovative and positive associations in the user, presents a pleasing feel when being worked into the hair, and is readily washed out.

The object of the present invention is a hair styling product that possesses a foamable or sprayable, gel-type emulsion and contains:
(A) at least one silicone compound, selected from among the esters of polyalkoxylated polydimethylsiloxanes;
(B) at least one gelling agent or thickener;
(C) at least one emulsifier; and
(D water
   in combination with a device for spraying or foaming the composition and their use for conditioning and/or for styling of human hair.

Foamable compositions are compositions from which a foam that is stable at least temporarily (e.g., at least 30 seconds) can be formed by means of suitable devices for foaming, such as, e.g., foam pumps or aerosol containers with a foaming head. Sprayable compositions are compositions from which a spray that has a sufficiently small droplet size (e.g., mean droplet size preferably smaller than 150 μm, particularly smaller than or equal to 100 μm) can be formed by means of suitable spraying devices such as, e.g., spray pumps or aerosol containers with a spraying head. A gel-type emulsion can, for example, have a fluid consistency (liquid gel). As a rule, gel-type compositions are different from liquids in that they exhibit elastic properties, in other words the elastic component of the storage modulus (elastic modulus) G′ exceeds the value of the loss modulus G″. At room temperature (25 C.), G′ is larger than G″ in least in one portion of the region (e.g., at 20 hz), preferably in the entire region of normal measurement frequencies such as from 0.01 hz to 40 hz, which can be measured, for example, with a Bohlin Rheometer CS system. The viscosity of a gel-type emulsion is preferably from 500 mPas to 20,000 mPas, and from 1,000 mPas to 15,000 mPas is especially preferred, as measured with a HAAKE VT-501 rotational viscometer, an SV-DIN gauging member at a temperature of 25° C., and a shear rate of 12.9 s⁻¹ (speed range B, speed step 5). Emulsions are dispersed systems with at least one hydrophilic phase and at least one hydrophobic phase that is insoluble therein.

Alkoxylated Silicone Esters (A)

The polyalkoxylated polydimethylsiloxane esters (A) are preferably contained in an amount of from 0.1% to 30% by weight, or from 0.2% to 20% by weight, and from 1% to 10% by weight is especially preferred.

Polyalkoxylated polydimethylsiloxanes possess one or more polyalkylene oxide terminal or side-groups, preferably polyethylene oxide (polyethylene glycol) and/or polypropylene oxide (polypropylene glycol). The degree of alkoxylation is preferably from 2 to 40, especially from 5 to 30, 7 to 20 or 10 to 15. The polyalkoxylated polydimethylsiloxanes are preferably esterified with organic carboxylic acids, especially with fatty acids. These fatty acids are preferably monocarboxylic acids, dicarboxylic acids or hydroxycarboxylic acids containing at least 8, and preferably from 10 C-atoms to 32 C-atoms. Especially preferred are the fatty acids from natural waxes, e.g., the fatty acids from beeswax or candelilla wax.

Suitable esters of polyalkoxylated polydimethylsiloxanes are, e.g., those with the INCI designations bis-PEG-12 Dimethicone Beeswax, bis-PEG-12 Dimethicone Candellilate, Dimethicone PEG-15 Acetate, Dimethicone PEG-8 Adipate, Dimethicone PEG-7 Avocadoate, Dimethicone PEG-8 Avocadoate, Dimethicone PEG-8 Beeswax, Dimethicone PEG-8 Benzoate, Dimethicone PEG-8 Borageate, Dimethicone PEG-7 Cocoate, Dimethicone PEG-7 Isostearate, Dimethicone PEG-8 Isostearate, Dimethicone PEG-7 Lactate, Dimethicone PEG-8 Lanolate, Dimethicone PEG-8 Laurate, Dimethicone PEG-8 Meadowfoamate, Dimethicone PEG-7 Octyldodecyl Citrate, Dimethicone PEG-7 Olivate, Dimethicone PEG-8 Olivate, Dimethicone PEG-7 Phthalate, Dimethicone PEG-8 Phthalate, Dimethicone PEG/PPG-20/23 Benzoate, Dimethicone PEG-8 Succinate, Dimethicone PEG-7 Undecylenate. Preferred are especially fatty acid esters of bis-(polyethylene oxide) polydimethylsiloxanes.

Thickener, Gelling Agents (B)

Thickener or gelling agents are preferably contained in component (B) in an amount of from 0.05% to 30% by weight, from 0.2% to 20% by weight, and from 0.5% to 10% by weight is especially preferred. Suitable thickeners or gelling agents are
Natural-based polymers, especially polysaccharides and their derivatives, e.g., sclerotium gum, starches, gelatins, celluloses and their derivative such as carboxymethylcellulose, hydroxypropyl cellulose, methylcellulose, hydroxypropylmethylcellulose or hydroxyethylcellulose, microcrystalline cellulose, as well as extracts from algae such as agar, carrageenan or algenate, as well as carouba gum, guar gum and their derivative such as, e.g., alkylated or hydroxyalkylated guar, karaya gum, xanthan gum, gum arabic, pectins, gellan gum
synthetic polymers such as, e.g., polyvinylpyrrolidone or crosslinked polyacrylates (carbomers, carbopols),
inorganic thickeners such as hectorites, bentonites, aluminum, and magnesium silicates,
   or a mixture of the named substances.

Preferred thickeners are polysaccharides or polysaccharide derivatives such as, e.g., carboxylates, alkyl ethers, hydroxyalkyl ethers, alkyl esters or hydroxyalkyl esters. Especially preferred is a content of from 0.1% to 10% by weight of carrageenan, especially kappa-carrageenan and/or iota-carrageenan as well as gellan gum.

Emulsifier (C)

The emulsifiers (C) can be present in amounts of from 0.1% to 30% by weight, preferably from 0.2% to 5% by weight. Nonionic, anionic, cationic, amphoteric or zwitterionic surfactants can be used. Suitable surfactants are for example the surfactants listed in the “International Cosmetic Ingredient Dictionary and Handbook,” 7th Ed., Volume 2 in the section on ‘Surfactants,’ and especially in the subsection on ‘Surfactants—Emulsifying Agents.’ Silicone surfactants are preferred, especially alkoxylated dimethylpolysiloxanes, as well as non-silicone-containing, nonionic organic surfactants.

Nonionic surfactant are, e.g., oxyethylated fatty alcohols, oxyethylated nonylphenols, alkylpolyglycosides, fatty acid mono- and diglycerides, ethoxylated and hydrogenated or non-hydrogenated castor oil, fatty acid alkanolamides, and oxyethylated fatty acid esters. Cationic surfactants are, e.g., long-chain quaternary ammonium compounds such as those known by the CTFA designation “Quaternium,” such as, e.g., alkyltrimethylammonium salts or dialkyldimethylammonium salts with from C-8 to C-22 alkyl groups. Suitable cationic surfactants are those represented by the generic formula

N⁽⁺⁾R¹R²R³R⁴X⁽⁻⁾

where R1 to R4 independently from one another indicate aliphatic groups, aromatic groups, alkoxy groups, polyoxyalkylene groups, alkylamido groups, hydroxyalkyl groups, aryl groups or alkaryl groups with respectively 1 C-atom to 22 C-atoms, and where at least one of the residues R1 to R4 possess at least 8 C-atoms and X⁽⁻⁾ represents a cosmetically compatible anion, e.g., a halide, acetate, phosphate, nitrate, or alkylsulfate, preferably a chloride. In addition to carbon atoms and hydrogen atoms, the aliphatic groups can also contain crosslinks or other groups, such as, e.g., further amino groups. Examples of suitable cationic surfactants are the chlorides or bromides of alkyldimethylbenzylammonium salts, alkyltrimethylammonium salts, e.g., cetyltrimethylammonium chloride or bromide, tetradecyltrimethylammonium chloride or bromide, alkyldimethylhydroxyethylammonium chlorides or bromides, dialkyldimethylammonium chlorides or bromides, alkylpyridinium salts, e.g., lauryl- or acetylpyridinium chloride, alkylamidoethyltrimethylammonium ether sulfates as well as compound with cationic character such as amine oxides, for example, alkylmethylamine oxides or alkylaminoethyldimethylamine oxides. Especially preferred is cetyltrimethylammonium chloride. Suitable cationic surfactants are in particular also the so-called ester quats, e.g., C-8 to C-18 alkyl esters of betaine, for example palmityl betaine chloride.

Anionic surfactants can be selected, for example, from among the alkaline or alkaline earth salts of C-10 to C-18 alkylsulfates, C-10 to C-18 alkylsulfonates, C-10 to C-18 alkylbenzenesulfonates, C-10 to C-18 xylenesulfonates and C-10 to C-18 alkyl ether sulfates ethoxylated with from 1 to 10 ethylene oxide units, ethoxylated sulfosuccinate halfesters of the generic formula

R—(OCH₂CH₂)ₘ—O₂C—CH₂—CH(SO₃M)—COOM

wherein R indicates a C-10 to C-18 alkyl residue, M represents an alkaline or alkaline earth cation and m indicates a number from 1 to 10, and alkyl ether carboxylates of the generic formula

R—(OCH₂CH₂)ₙ—OCH₂—COOM

wherein R indicates a C-10 to C-18 alkyl residue, M indicates an alkaline or alkaline earth cation and n indicates a number from 1 to 20, and where the alkaline and alkaline earth salts of C-10 to C-18 alkyl ether sulfates ethoxylated with 1 to 10 ethylene oxide units are especially preferred.

Amphoteric surfactants can be selected, for example, from among the derivatives of aliphatic quaternary ammonium, phosphonium, and sulfonium compounds of the generic formula

R¹—Y⁽⁺⁾(R²)—CH₂—R³—Z⁽⁻⁾

where R1 represents a linear or branched alkyl, alkenyl, or hydroxyalkyl group with from 8 C-atoms to 18 C-atoms and from 0 ethylene oxide units to 10 ethylene oxide units and from 0 glycerol units to 1 glycerol unit; Y is equal to an N-, P- or S-containing group; R2 is equal to an alkyl or monohydroxyalkyl group with from 1 C-atom to 3 C-atoms; x equals 1 when Y equals an S-atom and x equals 2 when Y equals an N- or P-atom; R3 is an alkyl or hydroxyalkyl group with from 1 C-atom to 4 C-atoms and Z represents a carboxylate, sulfate, phosphonate or phosphate group. Further suitable amphoteric surfactants are those that are derived from betaine, e.g., C-8 to C-18 alkyl betaines such as coco dimethylcarboxymethyl betaine, lauryl dimethylcarboxymethyl betaine, lauryl dimethyl-alpha-carboxyethyl betaine, cetyl dimethylcarboxymethyl betaine, oleyidimethyl-gamma-carboxypropyl betaine or lauryl bis-(2-hydroxypropyl)-alpha-carboxyethyl betaine; C-8 to C-18 alkyl sulfobetaines such as coco dimethylsulfopropyl betaine, stearyl dimethylsulfopropyl betaine, lauryl dimethylsulfoethyl betaine, lauryl bis-(2-hydroxyethyl)sulfopropyl betaine; carboxyl derivatives of imidazole, C-8 to C-18 alkyldimethylammonium acetates, C-8 to C-18 alkyldimethylcarbonylmethylammonium salts as well as C-8 to C-18 fatty acid alkylamido betaines, such as, for example, coconut fatty acid amidopropyl betaine (INCI designation: Ccocamidopropyl Betaine) and N-coconut fatty acid amidoethyl-N-[2-(carboxymethoxy)ethyl]glycerol (INCI designation: Cocoamphocarboxyglycinate).

Preferred silicone surfactants are alkoxylated dimethylpolysiloxanes. This example concerns siloxane/polyoxyalkylene copolymers. These are siloxanes with polyalkylene oxide groups, especially silicones that are modified with polypropylene oxide, polyethylene oxide or their mixtures. The respective alkylene oxide groups can occupy side-chain or terminal positions or they may be involved in linear polydimethylsiloxane/polyalkylene oxide block copolymers. The degree of alkoxylation is preferably from 2 to 40, especially from 10 to 30, and from 12 is 20 is especially preferred. The alkylene oxide-modified siloxanes are also known by the earlier INCI designation Dimethicone Copolyols.

Such silicone surfactants can be represented by the generic formula

A-Bₓ-D_{y}-A

where A stands for the monofunctional group R₂R¹SiO_{1/2}, B stands for the difunctional group R₂SiO, D stands for the difunctional group RR¹SiO, R independently from one another stands for H, C-1 to C-6 alkyl or aryl, preferably stands for H or C-1 to C-4 alkyl, and is especially preferred when it stands for methyl, R¹ stands for an oxyalkylene-containing group, hydrogen or methyl, x stands for a number from 10 to 1000, preferably from 10 to 500, and especially preferably from 20 to 200, and y stands for a number from 0 to 100, preferably from 1 to 50, provided that the compound contains at least one oxyalkylene-containing group R¹. The group -R¹ stands preferably for a group represented by the generic formula

-R²(OCₙH₂ₙ)ₘR³

where R² is a divalent group in which the oxyalkylene unit is bonded to the siloxane chain, preferably CₚH₂ₚ with p equal to 2 to 8, preferably 2 to 6, and especially preferably 3 to 6; R³ is a monofunctional end group for the oxyalkylene unit, for example H, OH, C-1 to C-6 alkyl, aryl, C-1 to C-6 alkoxy, C-1 to C-6 acyloxy, preferably OH; n is a number from 2 to 4, preferably 2 or 3, and m is a number that is at least 1, wherein the sum of the m for all oxyalkylene groups is 2 to 40, preferably 10 to 30.

Suitable silicone surfactants are available commercially, e.g., DC 3225 C, DC Q2-5220, DC 193, DC 190, or DC Q4-3667 from Dow Corning, Silwet® L-7200 from OSI Specialties, Abil® B8830, Abil® B8851, Abil® B8863, or Abil® EM97 from Goldschmidt, SF-1188 from General Electric or KF353A from Shin Etsu.

Preferred silicone compounds are those with the INCI designations
PEG-x Dimethicones, where x stands for the degree of ethoxylation and, for example, can have a value of 2 to 20, especially the values 3, 7, 8, 9, 10, 12, 14 or 17;
PPG-y Dimethicone, where y stands for the degree of propoxylation and, for example, can have a value of 2 to 30, especially the values 12 or 27;
and, PEG/PPG-x/y, where x stands for the degree of ethoxylation and y stands for the degree of propoxylation, and, for example, each of which can have values of 2 to 30, in particular can have the respective values 3/10, 4/12, 6/11, 8/14, 12/16, 12/18, 14/4, 15/15, 16/2, 16/8, 17/18, 18/18, 19/19, 20/6, 20/15, 20/20, 20/23, 20/29, 22/23, 22/24, 23/6, 25/25, and 27/27.

Preferred silicone surfactants are especially bis-alkoxylated silicone compounds, in other words poly(dialkylsiloxanes), that possess two terminal or side-chain polyoxyalkylene chains. Block copolymers are preferred, especially of the ABA type, with a middle block of polydimethylsiloxane and end-blocks of polyethylene oxide and/or polypropylene oxide. The end blocks can be substituted or preferably unsubstituted on the chain ends, in other words presenting free hydroxyl groups. The degree of alkoxylation is preferably from 2 to 40, especially from 10 to 30, especially preferably from 12 to 20.

Suitable silicone compounds are also those represented by the formula

R1—(AO)ₓ₁—B1—SiMe₂O—(SiMe₂O)ₓ₃—SiMe₂O—B2—(AO)ₓ₂—R2,

where R1 is a hydroxyl group, alkoxy group with 1 C-atom to 22 C-atoms or carboxyalkyl group with 2 C-atoms to 22 C-atoms, B1 and B2 can be different or preferably the same and can indicate a single bond or a divalent bonding group, especially an alkylene group with 1, 2, 3, or 4 C-atoms; AO indicates an oxyalkylene group, especially oxyethylene or oxypropylene; R2 is hydrogen or an alkyl group with from 1 C-atom to 22 C-atoms that is etherified or esterified with the neighboring oxyalkylene group; x1 and x2 are numbers greater than or equal to 1 and their sum provides the degree of alkoxylation and x3 is a number greater than or equal to 1 and provides the degree of polymerization of the dimethylpolysiloxane.

Preferred silicone compounds are those with the INCI designations
Bis-PEG-x Dimethicone, where x stands for the degree of ethoxylation and, for example, can have values of from 2 to 30, especially the values 4, 12, and 20;
Bis-PEG/PPG-x/y Dimethicone where x stands for the degree of ethoxylation and y stands for the degree of propoxylation and for example can each have values of from 2 to 30, especially the values 14/14, 20/20, and 6/16.

Especially preferred are bis-(polyethylene oxide) polydimethylsiloxanes, e.g., Bis-PEG-4 Dimethicone, Bis-PEG-12 Dimethicone, Bis-PEG-20 Dimethicone, Bis-PEG/PPG-14/14 Dimethicone, Bis-PEG/PPG-20/20 Dimethicone, Bis-PEG/PPG-16/16 PEG/PPG-16/16 Dimethicone.

Solvent

Water is contained in the emulsion as the hydrophilic solvent. The water content can be from 20% to 90% by weight, 30% to 80% by weight, or preferably 40% to 70% by weight. In addition to water, further water-soluble, cosmetically compatible, organic solvent can be contained in the hydrophilic phase, for example in amounts of from 1 to 40 percent by weight or 5% to 30% by weight. Such solvents are, for example, lower monohydric alcohol such as ethanol or isopropanol, or polyhydric C-2 to C-4 alcohols, such as, for example, ethylene glycol, diethylene glycol, butylene glycol, or glycerol.

Formulations for Application

One embodiment of the hair styling product of the present invention relates to an aerosol or non-aerosol foam product (mousse) together with a suitable device for foaming. The emulsion is thus foamable, in other words it contains at least one conventional, heretofore known foam-generating substance, e.g., at least one foam-producing surfactant and/or at least one foam-producing polymer. Devices for foaming are understood to be devices that enable the foaming of a liquid either with or without the use of an aerosol propellant. As a suitable mechanical foaming device, for example a commercially available foam pump or an aerosol foaming head can be used. A (foam pump) that is a non-aerosol foaming product will possess a mechanical pump foaming device. A aerosol foaming product will possess at least one aerosol propellant, a pressure-proof container and a device for foaming in the form of a foaming head. The aerosol propellant can be contained, for example, in an amount of from 1% to 20% by weight, from 2% to 15% by weight, or from 5% to 10% by weight. The aerosol propellant can be selected for example from among propane, butane, dimethylether and fluorinated hydrocarbons, where propane and butane are preferred. The agent is foamed immediately before use and is worked into the hair as a foam and can then be rinsed out or can be left in the hair without rinsing.

A further embodiment of the hair styling product of the present invention relates to a pump-spray product and comprises a mechanical pump spraying device. The emulsion can be sprayed by means of a suitable mechanically driven spraying device. A mechanical spraying device is understood to be a device that enables the spraying of a composition without the use of a aerosol propellant. As suitable mechanical spraying devices, for example, a spray pump or an elastic container with a spray valve can be used, which would be filled under pressure with a cosmetic agent of the present invention, during the process of which the elastic container will undergo expansion so that the agent will be continuously expelled from the elastic container by contraction of same when the spray valve has been opened.

Hair-Setting and Hair-Conditioning Polymers

In one embodiment, the composition of the present invention contains at least one hair-setting and/or at least one hair-conditioning polymer. The hair-setting or hair-conditioning polymers are preferably contained in an amount of from 0.01% to 20% by weight or from 0.05% to 10% by weight, especially preferably from 0.1% to 5% by weight. These polymers can be anionic polymers, in other words polymers with anionic or anionizable groups, or cationic polymers, in other words polymers with cationic or cationizable groups, or zwitterionic polymers, in other words polymers with cationic and anionic group, or amphoteric polymers, in other words polymers with acidic and basic groups, or nonionic polymers. Anionizable groups are understood to be acid groups, such as, for example, carboxylic acid, sulfonic acid, or phosphoric acid groups, which can be deprotonated by means of conventional bases, such as, for example, organic amines or alkaline or alkaline earth hydroxides.

Anionic polymers can be partially or completely neutralized with a basic neutralizing agent. A preferred degree of neutralization is from 50% to 100%, especially preferably from 70% to 100%. An organic or inorganic base can be used as the neutralizing agent. Particular examples of bases are aminoalkanols, such as, for example, aminomethylpropanol (AMP), triethanolamine or monoethanolamine, and also ammonia, NaOH, KOH among others.

The anionic polymer can be a homo- or copolymer with acid group-containing monomer units derived from natural or synthetic sources, which if necessary can be polymerized with comonomers that contain no acid groups. Among the acid group that can be considered are sulfonic acid, phosphoric acid, and carboxylic acid groups, of which the carboxylic acid groups are preferred. Suitable acid group-containing monomer are for example acrylic acid, methacrylic acid, crotonic acid, maleic acid, and maleic anhydride, maleic acid monoesters, especially the C-1 to C-7 alkyl monoesters of maleic acid as well as aldehydocarboxylic acids or ketocarboxylic acids. Comonomers that are not substituted with acid groups are, for example, acrylamide, methacrylamide, alkyl- and dialkylacrylamide, alkyl- and dialkylmethacrylamide, alkyl acrylate, alkyl methacrylate, vinylcaprolactone, vinylpyrrolidone, vinyl ester, vinyl alcohol, propylene glycol or ethylene glycol, amine-substituted vinyl monomers, such as, for example, dialkylaminoalkyl acrylate, dialkylaminoalkyl methacrylate, monoalkylaminoalkyl acrylate and monoalkylaminoalkyl methacrylate, where the alkyl groups of these monomer are preferably C-1 to C-7 alkyl group, especially preferably C-1 to C-3 alkyl groups.

Suitable polymers with acid groups are especially homopolymers of acrylic acid or methacrylic acid, copolymers of acrylic acid or methacrylic acid with monomers selected from acrylic acid or methacrylic acid esters, acrylamides, methacrylamides and vinylpyrrolidone, homopolymers of crotonic acid as well as copolymer of crotonic acid with monomers selected from vinyl esters, acrylic acid or methacrylic acid esters, acrylamides and methacrylamides that are uncrosslinked or crosslinked with polyfunctional agents. A suitable natural polymer is for example schellac.

Preferred polymers with acid groups are: Terpolymers from acrylic acid, alkyl acrylate and N-alkylacrylamide (INCI designation: Acrylate/Acrylamide Copolymer), especially terpolymers from acrylic acid, ethyl acrylate and N-tert-butylacrylamide; crosslinked or uncrosslinked vinyl acetate/crotonic acid copolymers (INCI designation: VA/Crotonate Copolymer); copolymers from one or more C-1 to C-5 alkyl acrylates, especially C-2 to C-4 alkyl acrylates and at least one monomer selected from acrylic acid or methacrylic acid (INCI designation: Acrylate Copolymer), e.g., terpolymers from tert-butyl acrylate, ethyl acrylate and methacrylic acid; sodium polystyrenesulfonate; vinylacetate/crotonic acid/vinyl alkanoate copolymers, for example, copolymers from vinyl acetate, crotonic acid and vinyl propionate; copolymers from vinyl acetate, crotonic acid and vinyl neodecanoate (INCI designations: VA/ Crotonate/Vinyl Propionate Copolymer, VA/Crotonate/Vinyl Neodecanoate Copolymer); aminomethylpropanol acrylate copolymers; copolymers from vinylpyrrolidone and at least one further monomer selected from acrylic acid and methacrylic acid as well as if necessaryAcrylic acid esters and methacrylic acid esters; copolymers from methyl vinyl ether and maleic acid monoalkylesters (INCI designations: Ethyl Ester of PVM/MA Copolymer, Butyl Ester of PVM/MA Copolymer); aminomethylpropanol salts of copolymers from allyl methacrylate and at least one further monomer selected from acrylic acid, and methacrylic acid as well as if necessaryAcrylic acid esters and methacrylic acid esters; crosslinked copolymers from ethyl acrylate and methacrylic acid; copolymers from vinyl acetate, mono-n-butyl maleate and isobornyl acrylate; copolymers from two or more monomers selected from acrylic acid and methacrylic acid as well as if necessary Acrylic acid esters and methacrylic acid esters; copolymers from octylacrylamide and at least one monomer selected from acrylic acid and methacrylic acid as well as if necessary Acrylic acid esters and methacrylic acid esters; polyesters from diglycol, cyclohexanedimethanol, isophthalic acid and sulfoisophthalic acid, where the alkyl groups of the aforementioned polymers as a rule preferably possess 1, 2, 3, or 4 C-atoms.

Preferred zwitterionic or amphoteric polymers are: copolymers formed from alkylacrylamide, alkylaminoalkyl methacrylate and two or more monomer from acrylic acid and methacrylic acid as well as if necessary their esters, especially copolymers from octylacrylamide, acrylic acid, butylaminoethyl methacrylate, methyl methacrylate and hydroxypropyl methacrylate (INCI designation: octylacrylamide/acrylatelbutylaminoethyl methacrylate copolymer); copolymers, that are formed from at least one of a first type of monomer that possesses quaternary amino groups and at least one of a second type of monomer that possesses acid groups; copolymers from fatty alcohol acrylates, alkylamine oxide methacrylate and at least one monomer selected from acrylic acid and methacrylic acid as well as if necessary acrylic acid esters and methacrylic acid esters, especially copolymers from lauryl acrylate, stearyl acrylate, ethylamine oxide methacrylate and at least one monomer selected from acrylic acid and methacrylic acid as well as if necessary their esters; copolymers from methacryloyl ethyl betaine and at least one monomer selected from methacrylic acid and methacrylic acid ester; copolymers from acrylic acid, methyl acrylate and methacrylamidopropyltrimethylammonium chloride (INCI designation: Polyquaternium-47); copolymers from acrylamidopropyltrimethylammonium chloride and acrylates or copolymers from acrylamide, acrylamidopropyltrimethylammonium chloride, 2-amidopropylacrylamide sulfonate and dimethylaminopropylamine (INCI designation: Polyquaternium-43); oligomers or polymers, preparable from quaternary crotonoylbetaines or quaternary crotonoylbetaine esters.

Cationic polymers are especially those with primary, secondary, tertiary or quaternary amino groups. The cationic charge density will be preferably from 1 meq/g to 7 meq/g. Suitable cationic polymers preferably contain quaternary amino groups. Cationic polymers can be homo- or copolymers, where the quaternary nitrogen groups are contained either in the polymer chain or preferably as substituents on one or more of the monomers. The ammonium group-containing monomers can be copolymerized with non-cationic monomers. Suitable cationic monomer are unsaturated compounds that can undergo radical polymerization, which bear at least one cationic group, especially ammonium-substituted vinyl monomers, such as, for example, trialkylmethacryloxyalkylammonium, trialkylacryloxyalkylammonium, dialkyldiallylammonium and quaternary vinylammonium monomers with cyclic, cationic nitrogen-containing groups such as pyridinium, imidazolium or quaternary pyrrolidones, e.g., alkylvinylimidazolium, alkylvinylpyridinium, or alkylvinylpyrrolidone salts. The alkyl groups of these monomers are preferably lower alkyl groups, such as, for example, C-1 to C-7 alkyl groups, and especially preferred are C-1 to C-3 alkyl groups.

The ammonium group-containing monomers can be copolymerized with non-cationic monomers. Suitable comonomers are, for example, acrylamide, methacrylamide, alkyl- and dialkylacrylamide, alkyl- and dialkylmethacrylamide, alkyl acrylate, alkyl methacrylate, vinylcaprolactone, vinylcaprolactam, vinylpyrrolidone, vinyl esters, for example, vinyl acetate, vinyl alcohol, propylene glycol or ethylene glycol, where the alkyl groups of these monomers are preferably C-1 to C-7 alkyl groups, and especially preferred are C-1 to C-3 alkyl groups.

Suitable polymers with quaternary amino groups are, for example, those described in the CTFA Cosmetic Ingredient Dictionary under the designations Polyquaternium such as methylvinylimidazolium chloride/vinylpyrrolidone copolymer (Polyquaternium-16) or quaternized vinylpyrrolidone/dimethylaminoethyl methacrylate copolymer (Polyquaternium-1 1) as well as quaternary silicone polymers as well as oligomers, such as, for example, silicone polymers with quaternary end groups (Quaternium-80).

Preferred cationic polymers of synthetic origin: poly(dimethyldiallylammonium chloride); copolymers from acrylamide and dimethyldiallylammonium chloride; quaternary ammonium polymers, formed by the reaction of diethyl sulfate with a copolymer from vinylpyrrolidone and dimethylaminoethyl methacrylate, especially vinylpyrrolidone/dimethylaminoethyl methacrylate methosulfate copolymer (e.g., Gafquat® 755 N, Gafquat® 734); quaternary ammonium polymers from methylvinylimidazolium chloride and vinylpyrrolidone (e.g., LUVIQUAT® HM 550); Polyquaternium-35; Polyquaternium-57; polymers from trimethylammonium ethyl methacrylate chloride; terpolymers from dimethyldiallylammonium chloride, sodium acrylate and acrylamide (e.g., Merquat® Plus 3300); copolymers from vinylpyrrolidone, dimethylaminopropyl methacrylamide and methacryloylaminopropyllauryldimethylammonium chloride; terpolymers from vinylpyrrolidone, dimethylaminoethyl methacrylate and vinylcaprolactam (e.g., Gaffix® VC 713); vinylpyrrolidone/methacrylamidopropyltrimethylammonium chloride copolymers (e.g., Gafquat® HS 100); copolymers from vinylpyrrolidone and dimethylaminoethyl methacrylate; copolymers from vinylpyrrolidone, vinylcaprolactam and dimethylaminopropylacrylamide; poly- or oligoesters formed from at least one first type of monomer, that is selected from hydroxyacids substituted with at least one quaternary ammonium group; dimethylpolysiloxane substituted with quaternary ammonium groups in the terminal positions.

Suitable cationic polymers that are derived from natural sources are especially cationic derivatives of polysaccharides, for example, cationic derivatives of cellulose, starch or guar. Furthermore, chitosan and chitosan derivatives are suitable. Cationic polysaccharides have for example are represented by the generic formula

G-O-B-N⁺R^{a}R^{b}R^{c}X⁻

G is an anhydroglucose residue, for example, starch or cellulose anhydroglucoses; B is a divalent bonding group, for example, alkylene, oxyalkylene, polyoxyalkylene or hydroxyalkylene; R^{a}, R^{b}, and R^{c} are independent from one another alkyl, aryl, alkylaryl, arylalkyl, alkoxyalkyl, or alkoxyaryl any of which can have up to 18 C-atoms, where the total number of C-atoms in R^{a}, R^{b}, and R^{c} is preferably a maximum of 20; X is a conventional counteranion, for example, a halogen, acetate, phosphate, nitrate, or alkylsulfate, preferably a chloride. Cationic celluloses are, for example, those with the INCI designations Polyquaternium-10 or Polyquaternium-24. A suitable cationic guar derivative has, for example, the INCI designation Guar Hydroxypropyltrimonium Chloride.

Especially preferred cationically-active substances are chitosan, chitosan salts, and chitosan derivatives. Chitosans that can be used in the present invention can be fully or partially deacetylated chitins. By way of example, the molecular weight can be distributed over a broad range, from 20,000 g/mol to 5,000,000 g/mol, for example, from 30,000 g/mol to 70,000 g/mol. However, the molecular weight will preferably lie above 100,000 g/mol, and especially preferred is from 200,000 g/mol to 700,000 g/mol. The degree of deacetylation is preferably from 10% to 99%, and especially preferably from 60% to 99%. A preferred chitosan salt is chitosonium pyrrolidone carboxylate, e.g., Kytamer® PC with a molecular weight of from 200,000 g/mol to 300,000 g/mol and a degree of deacetylation of from 70% to 85%. Chitosan derivatives that can be considered include quaternized, alkylated or hydroxyalkylated derivatives, e.g., hydroxyethyl, hydroxypropyl or hydroxybutyl chitosan. The chitosans or chitosan derivatives are preferably present in their neutralized or partially neutralized form. The degree of neutralization will be preferably at least 50%, especially preferably between 70% and 100%, as calculated on the basis of the number of free basic groups. For the neutralization agent, in principle any cosmetically compatible inorganic or organic acid can be used, such as, for example, formic acid, tartaric acid, malic acid, lactic acid, citric acid, pyrrolidonecarboxylic acid, hydrochloric acid and the like, of which pyrrolidonecarboxylic acid is especially preferred.

Preferred cationic polymers derived from natural sources: cationic cellulose derivatives from hydroxyethylcellulose and diallyldimethylammonium chloride; cationic cellulose derivatives from hydroxyethylcellulose and with a trimethylammonium-substituted epoxides; chitosan and its salts; hydroxyalkylchitosans and their salts; alkylhydroxyalkylchitosans and their salts; N-hydroxyalkylchitosan alkyl ethers; N-hydroxyalkylchitosan benzyl ether.

Suitable nonionic polymers are homo- or copolymers, that are formed from at least one of the following monomers: vinylpyrrolidone, vinylcaprolactam, vinyl esters, such as, for example, vinyl acetate, vinyl alcohol, acrylamide, methacrylamide, alkyl- and dialkylacrylamide, alkyl- and dialkylmethacrylamide, alkyl acrylate, alkyl methacrylate, propylene glycol or ethylene glycol, where the alkyl groups in these monomers are preferably C-1 to C-7 alkyl groups, and especially preferred are C-1 to C-3 alkyl groups. Suitable homopolymers are, for example, those of vinylcaprolactam, vinylpyrrolidone or N-vinylformamide. Further suitable synthetic, film-forming, nonionic, hair-setting polymers are for example copolymerides from vinylpyrrolidone and vinyl acetate, terpolymers from vinylpyrrolidone, vinyl acetate and vinyl propionate, polyacrylamides; polyvinyl alcohols as well as polyethylene glycol/polypropylene glycol copolymers. Suitable natural, film-forming polymers are for example cellulose derivatives, e.g., hydroxyalkyl cellulose.

Preferred nonionic polymers are: polyvinylpyrrolidone, polyvinylcaprolactam, vinylpyrrolidone/vinyl acetate copolymers, polyvinylalcohol, isobutylene/ethylmaleimide/hydroxyethylmaleimide copolymer; copolymers from vinylpyrrolidone, vinyl acetate, and vinyl propionate.

Fats, Oils, Waxes

In one embodiment, the hydrophobic phase of the emulsion of the composition of the present invention contains at least one additional hydrophobic fatty, waxy, or oily substance. The additional hydrophobic substances are preferably contained in amounts of from 0.5% to 20% by weight or from 1% to 10% by weight. Especially preferred are mineral oils, fatty alcohols, and fatty acid triglycerides.

Suitable fats and waxes or wax-type substances will exhibit a solidification or drop point that is greater than or equal to 25° C., and will preferably lie in the range of from 30° C. to 100° C., especially in the range of from 40° C. to 90° C. Fats and wax-type substances are, for example, animal waxes, vegetable waxes, mineral waxes, synthetic waxes, microcrystalline waxes, macrocrystalline waxes, paraffin waxes, ozocerite, montan waxes, Fischer-Tropsch waxes, polyolefin waxes (e.g., polyethylene, polybutene and the like), amide waxes, silicone waxes, beeswax, wool wax (lanolin) and its derivatives, such as, for example, wool wax alcohols (lanolin alcohols), candelilla wax, carnauba wax, Japan wax, fats, fatty acid esters, fatty acid glycerides, long-chain carboxylic acids or long-chain (C-10 to C-22) alcohols, each of which will have a melting, solidification or drop point above 25° C. For example, castor oil wax is suitable when is hardened, in other words when it is hydrogenated castor oil (INCI: Hydrogenated Castor Oil) with a melting point up to approximately 90° C.

Suitable oily substances are hydrophobic substances that are liquid at room temperature. This includes oils or oil-type substances, such as, for example, naturally-occurring, renewable oils (vegetable and animal fatty oils), synthetic oils, silicone oils, especially linear or cyclic dimethylpolysiloxanes, mineral oils, essential oils, water-insoluble, branched or linear aliphatic hydrocarbons, linear or branched alcohols, especially liquid fatty alcohols such as long-chain ethers or esters, where the named substances preferably possess at least 8 C-atoms. Suitable hydrocarbons are, for example, liquid paraffins, squalanes, or squalenes. Furthermore suitable are esters of tri- and polyhydric alcohols, especially vegetable triglyceride, such as, for example, olive oil, almond oil, peanut oil, sunflower oil as well as synthetic triglycerides, such as, for example, C-8 to C-10 tri-fatty acid glycerol esters or jojoba oil.

Additional suitable hydrophobic substances are mono- or diesters of the formulas R¹—COOR², R¹—COO—R³—OOCR¹, and R²OOC—R³—COOR², where R¹ stands for a C-8 to C-22 alkyl group, R² stands for a C-3 to C-22 alkyl group and R³ stands for a C-2 to C-16 alkylene group. Also suitable are naturally-occurring monoesters or wax ester mixtures, such as are present for example in jojoba oil or sperm oil and branched primary alcohols, which are known by the designation Guerbet alcohols. Additional substances that are suitable as hydrophobic substances are those that are commonly introduced in cosmetic agents as opacifying agents, especially those represented by the formula R¹—COO—(CHR⁴CHR⁵O)ₙ—COR⁶, where R¹ stands for a C-8 to C-22 alkyl group, R⁴ and R⁵ stand for hydrogen or methyl and R⁶ stands for hydrogen or for R¹ and n indicates a number between 1 and 12, preferably 1, 2, 3 or 4. Glycol di-fatty acid esters and polyethylene glycol di-fatty acid esters are preferred, which exist in solid form at room temperature.

Additives

The products of the present invention can contain additional active substances and adjuvants in the emulsion. The additional active substances and adjuvants are preferably contained for example in amounts of from 0.01% to 10% by weight, especially preferably from 0.1% to 5% by weight. The additives can be selected, for example, from among plant and herb extracts, protein and silk hydrolysates, photoprotective agents, antioxidants, radical scavengers, anti-flaking agents, glazing agents, vitamins, panthenol, softeners, combability-improving agents, proteins, bactericides, virucides, antimicrobials, proteolytically- or keratolytically-active substances, keratin-reducing substances, oxidizing substances, direct-penetrating hair dyes, and oxidative dye precursors.

Preparation

The preparation of the emulsion can be carried out according to one of the known procedures. A review of modern procedures for the preparation of semisolid and liquid emulsions can be found in the article in the SÖFW-Journal, Volume 124 (5/98), pp 308-313 as well as in the article in the SÖFW-Journal, Volume 118. (5/92) pp 287-296. As a rule, the preparation of the emulsion takes place in such a manner that the hydrophobic phase is heated to 60° C. to 75° C. and the solid waxy substances are melted. Thickeners, gelling agents and if necessary styling polymers are swollen or dissolved in the aqueous phase and are likewise warmed to 60° C. to 75° C. The hot premixtures are combined and homogenized with a mixer or homogenizer. Temperature-sensitive or volatile components are added after cooling to 30° C. to 40° C. Following this, the completed emulsion is cooled to ambient temperature. Aerosol products are charged for example with from 5% to 10% by weight of propane/butane or dimethylether.

The products of the present invention can be used on both dry and moist hair, and disperse well into the hair. Hair that is treated with the products of the present invention will exhibit a pleasing waxy feel after drying, have the potential for restyling and above all will have good combability with moist hair when compared with conventional hair wax products. Moreover, the products are readily washed out at a later time. The foam produced by the foam products of the present invention is characterized by an especially pleasant, creamy feel during use when compared with conventional aerosol styling foams or hair conditioning foams.

With the cosmetic agent of the present invention, the quantity used will depend upon the amount and condition of the hair to be treated and will be a quantity sufficient to obtain the desired hair styling effect (typically 3 g-10 g), and is applied to the washed hair as a foam or spray and is spread in. Preferably the agent remains in the hair and becomes dry after use. The object of the invention is thus also a method for hair treatment, wherein
by means of the above described product of the present invention, a composition is first applied to the hand either as a spray or as a foam and is spread into the hair by hand, or is applied directly to the hair as a spray or a foam and is then spread in by hand, where said product can be used either on freshly washed, moist hair or on dry hair;
the composition applied to the hair is allowed to remain in the hair; and
then the desired hair style is produced.

The product of the present invention can be used in particular for the conditioning and/or for the styling of human hair.

The following examples should serve to illustrate further the object of the present invention.

### EXAMPLES

Hair styling products

| Raw ingredients | Ex. 1 | Ex. 2 | Ex. 3 |
|---|---|---|---|
| Bis-PEG-12 Dimethicone Beeswax | 4.5 | 3 | 4.5 |
| (Siliconyl Beeswax) | | | |
| Bis-PEG-12 Dimethicone Candelillate | 4.5 | 0.75 | 4.5 |
| (Siliconyl Candelilla) | | | |
| Kappa Carrageenan (Genugel ® X-901-02) | 0.2 | 0.4 | — |
| Iota-Carrageenan (Seaspen ® PF) | 0.6 | — | 0.6 |
| PEG-12 Dimethicone | 4.5 | 4.5 | 4.5 |
| PEG-25 Hydrogenated Castor Oil | 4.5 | 1.5 | 4.5 |
| Vinylpyrrolidone/Methacrylamide/ | 7 | — | 7 |
| Vinylimidazole Copolymer | | | |
| (Luviset ® Clear) | | | |
| Vinylpyrrolidone/Vinyl Acetate Copolymer | 0.6 | — | 0.6 |
| (Luviskol ® VA 64) | | | |
| Vinyl Acetate/Crotonate/Vinyl Neodecanoate | — | 2.25 | — |
| Copolymer (Resyn 28-2930) | | | |
| Paraffinum Perliquidum | 1.5 | — | 1.5 |
| Adeps Lanae (Lanolin) | — | 3 | — |
| Shea Butter | 5 | — | 5 |
| Perfume | 0.2 | — | 0.2 |
| Aminomethylpropanol | — | 0.4 | — |
| Ethanol | 15 | 25 | 15 |
| Water | balance | balance | balance |
| | to 100 | to 100 | to 100 |

The preparation is carried out by melting the wax phase together with the oil components; swelling and dissolution of the thickeners and styling polymers in the aqueous phase; combining of the hot premixtures and homogenization, followed by addition of the temperature-sensitive or volatile components at 35° C. For aerosol foams, the emulsions are charged with 5% to 10% of propane/butane or dimethylether in pressure-proof containers with a foaming head. For pump sprays, the emulsions are packaged in suitable containers with spray pumps.

The aerosol foam is characterized by a unique foam quality. After being drawn out of the pressurized container, it develops into a visually pleasing foam. While being spread into the hair with the hands, it promptly develops into a creamy consistency that is spread readily. This material is optimally suited for spreading through the hair. Immediately after application, the product exhibits a high degree of tackiness in the hair, which improves the styling properties as compared with conventional styling waxes, foam waxes and foam setting preparations. The tackiness subsides automatically with increasing dryness, and the hair takes on a dry, waxy feel without having a greasy shine. The product provides the hair with excellent stability and very good styling properties.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair styling product comprising a foamable or sprayable, gel-type emulsion that comprises:
(A) at least one silicone compound, selected from esters of polyalkoxylated polydimethylsiloxanes;
(B) at least one gelling agent or thickener;
(C) at least one emulsifier; and
(D) water
in combination with a device for the spraying or foaming of the composition.

2. A product according to claim 1 wherein the emulsion comprises said silicone compounds in an amount of from 0.1% to 30% by weight, said gelling agents or thickeners in an amount of from 0.05% to 30% by weight, emulsifiers in an amount of from 0.1% to 30% by weight and/or water in an amount of from 20% to 90% by weight.

3. A product according to claim 1 wherein the silicone compound is selected from among esters of polyethoxylated polydimethylsiloxanes with fatty acids possessing at least 8 C-atoms.

4. A product according to claim I wherein the gelling agents or thickeners are selected from the group consisting of
polymers from natural sources, selected from the group consisting of carrageenans, gellan gum, sclerotium gum, starches, gelatins, cellulose, carboxymethylcellulose, hydroxypropyl cellulose, methylcellulose, hydroxypropylmethylcellulose, hydroxyethylcellulose, microcrystalline cellulose, agar, algenates, carouba gum, guar gum, alkylated guar, hydroxyalkylated guar, karaya gum, xanthan gum, gum arabic and pectin;
synthetic polymers, selected from the group consisting of polyvinylpyrrolidone and crosslinked polyacrylates; and
inorganic thickeners, selected from the group consisting of hectorites, bentonites, aluminum silicates, and magnesium silicates.

5. A product according to claim 4 comprising the carrageenan in an amount of from 0.1% to 10% by weight.

6. A product according to claim 1 wherein the emulsifier is selected from the group consisting of silicone surfactants and nonionic, non-silicon-containing organic surfactants.

7. A product according to claim 1 wherein said product further comprises at least one aerosol propellant, the emulsion is foamable and the product comprises a device for foaming.

8. A product according to claim 1 wherein said product is free from aerosol propellants, the emulsion is sprayable and the product comprises a mechanical device for spraying.

9. A product according to claim 1 wherein the emulsion further comprises at least one hair-setting and/or hair-conditioning polymer.

10. A product according to claim 9 wherein the hair-setting and/or hair-conditioning polymer is present in an amount of from 0.01% to 20% by weight and is selected from the group consisting of
polymers with anionic or anionizable group, selected from the group consisting of terpolymers from acrylic acid, ethyl acrylate, N-tert-butylacrylamide, crosslinked or uncrosslinked vinyl acetate/crotonic acid copolymers, terpolymers from tert-butylacrylate, ethyl acrylate, methacrylic acid, sodium polystyrenesulfonate, copolymers from vinyl acetate, crotonic acid, vinyl propionate, copolymers from vinyl acetate, crotonic acid, vinyl neodecanoate, aminomethylpropanol/acrylate copolymers, copolymers from vinylpyrrolidone, acrylic acid, methacrylic acid, acrylic acid esters, methacrylic acid esters, copolymers from methyl vinyl ether, maleic acid monoalkyl esters, aminomethylpropanol salts of copolymers from allyl methacrylate, crosslinked copolymers from ethyl acrylate, crosslinked copolymers from methacrylic acid, copolymers from vinyl acetate, mono-n-butyl maleate, isobornyl acrylate, copolymers from two or more monomers selected from among acrylic acid, methacrylic acid, acrylic acid esters and methacrylic acid esters, copolymers from octylacrylamide and at least one monomer selected from among acrylic acid, methacrylic acid, acrylic acid esters and methacrylic acid esters; polyesters from diglycol, cyclohexanedimethanol, isophthalic acid, and sulfoisophthalic acid;
polymers with cationic or cationizable group, selected from among cationic cellulose derivatives from hydroxyethylcellulose and diallyldimethylammonium chloride; cationic cellulose derivatives from hydroxyethylcellulose and with trimethylammonium substituted epoxides; poly(dimethyldiallylammonium chloride); copolymers from acrylamide and dimethyldiallylammonium chloride; quaternary ammonium polymers, formed from the reaction of diethyl sulfate with a copolymer from vinylpyrrolidone and dimethylaminoethyl methacrylate; quaternary ammonium polymers from methylvinylimidazolium chloride and vinylpyrrolidone; Polyquaternium-35; polymers from trimethylammoniumethyl methacrylate chloride; Polyquaternium-57; dimethylpolysiloxanes substituted with quaternary ammonium groups at the terminal positions; copolymers from vinylpyrrolidone, dimethylaminopropyl methacrylamide and methacryloylaminopropyllauryldimethylammonium chloride; chitosan and its salts; hydroxyalkylchitosans and their salts; alkylhydroxyalkylchitosans and their salts; N-hydroxyalkylchitosan alkyl ethers; N-hydroxyalkylchitosan benzyl ethers; copolymers from vinylcaprolactam, vinylpyrrolidone and dimethylaminoethyl methacrylate; copolymers from vinylpyrrolidone and dimethylaminoethyl methacrylate, copolymers from vinylpyrrolidone, vinylcaprolactam and dimethylaminopropyl acrylamide; poly- or oligoesters formed from at least one first type of monomer that is selected from among hydroxyacids that are substituted with at least one quaternary ammonium group; terpolymers from vinylpyrrolidone, methacrylamide, and vinylimidazole;
zwitterionic and/or amphoteric polymers, selected from among copolymers from octyl acrylamide, acrylic acid, butylaminoethyl methacrylate, methyl methacrylate and hydroxypropyl methacrylate; copolymers from lauryl acrylate, stearyl acrylate, ethylamine oxide methacrylate and at least one monomer selected from among acrylic acid, methacrylic acid, acrylic acid esters and methacrylic acid esters; copolymers from methacryloyl ethyl betaine and at least one monomer selected from among methacrylic acid and methacrylic acid esters; copolymer from acrylic acid, methyl acrylate and methacrylamidopropyltrimethylammonium chloride; oligomers or polymers that can be prepared from quaternary crotonoylbetaines or quaternary crotonoylbetaine esters;
nonionic polymers, selected from among polyvinylpyrrolidone, polyvinylcaprolactam, vinylpyrrolidone/vinyl acetate copolymers, polyvinylalcohol, isobutylene/ethyl maleimide/hydroxyethyl maleimide copolymer; copolymers from vinylpyrrolidone, vinyl acetate, and vinylpropionate.

11. A product according to claim 1 wherein the emulsion further comprises at least one further hydrophobic fatty, waxy, or oily substance.

12. A product according to claim 11 wherein the further hydrophobic fatty, waxy or oily substance comprises an amount of from 0.5% to 20% by weight and is selected from among mineral oils, fatty alcohols, and fatty acid triglycerides.

13. A product according to claim 1 wherein the emulsion comprises at least one mono- or polyhydric alcohol having from 1 C-atom to 5 C-atoms.

14. A product according to claim 13 wherein ethanol comprises as the alcohol in an amount of from 5% to 40% by weight.

15. A use of the product according to claim 1 for the hair conditioning or hair styling of human hair.

16. A method for hair treatment wherein
by using a product according to claim 1 the composition as a spray or a foam is either first applied to the hand and then spread with the hand into the hair or is directly applied to the hair and from there is spread by the hand, where the application can take place either on freshly washed, moist hair or on dry hair;
the composition applied to the hair is allowed to remain on the hair; and
then the desired hair style is produced.
